(19) United States
(12) Patent Application Publication
(10) Pub. No.: US 2025/0228617 A1
(43) Pub. Date: Jul. 17, 2025

(54) COMPUTATIONAL BASED 3D MODELING METHODS AND SYSTEMS FOR ASSISTING TRANSCATHETER AORTIC VALVE REPLACEMENT (TAVR) PROCEDURES

(71) Applicant: The Regents of the University of Colorado, a body corporate, Denver, CO (US)

(72) Inventors: Shiuh-Yung J. CHEN, Denver, CO (US); John D. CARROLL, Denver, CO (US); Robert A. QUAIFE, Denver, CO (US); John C. MESSENGER, Denver, CO (US)

(21) Appl. No.: 18/727,980

(22) PCT Filed: Jan. 10, 2023
(86) PCT No.: PCT/US2023/060401

§ 371 Date: Jul. 10, 2024

(51) Int. Cl. A61B 34/10; A61F 2/24; G06T 7/00; G06T 7/11; G06T 7/174; G06T 15/08; G06T 15/20

(52) U.S. Cl. CPC A61B 34/10; G06T 7/0016; G06T 7/11; G06T 7/174; G06T 15/08; G06T 15/20; A61B 2034/105; A61B 2034/107; A61F 2/2427; G06T 2207/10081; G06T 2207/30048; G06T 2207/30104; G06T 2210/41; G06T 2211/404

(57) ABSTRACT

Noninvasive imaging plays an important role in determining the size of the transcatheter heart valve (THV) in preparation of a transcatheter aortic valve replacement (TAVR) procedure. The identification of the coronary cuspid landmarks or nadirs of each cusp plays a key role in determining the reference points for the two-dimensional (2D) images of the THV. Rather than using nadirs that are currently identified manually upon inspection of 2D computed tomography (CT) images, the methods and systems of the present disclosure utilizing structured 3D dataset of cusps to simultaneously determine the nadirs, which are especially beneficial, particularly when the sizes of cusps have significant difference (i.e., Type 1 aortic valve with two fused leaflets) or the aortic root has a bicuspid configuration rather than a tricuspid configuration.

### TECHNICAL FIELD

The present disclosure relates generally to systems and methods for performing a transcatheter aortic valve replacement and/or preparing to perform a transcatheter aortic valve replacement. More particularly, the present disclosure relates to a method and system for determining the size of the aortic valve and guiding proper implantation based on an X-ray with C-arm gantry imaging system.

### BACKGROUND

Aortic valve stenosis occurs when the heart's aortic valve thickens and calcifies, thereby preventing the aortic valve from opening fully, which limits blood flow from the heart to the rest of body. Transcatheter aortic valve replacement (TAVR) is a minimally invasive procedure that addresses aortic valve stenosis by replacing the aortic valve that fails to open properly with an implantable transcatheter heart valve (THV). To perform a TAVR procedure, an interventionist accesses a patient's heart through a blood vessel in the leg or through an incision in the chest, thereby creating an access point. A catheter is inserted through the access point, navigates blood vessels to the heart and enters the aortic valve. Once the new THV is at the desired location, a balloon on the catheter's tip is inflated to expand the replacement THV into the appropriate position. Alternatively, some valves made of self-expanding materials (i.e., nitinol alloy) can expand without the use of a balloon after being deployed from the catheter.

Noninvasive imaging plays an important role in determining the size of the THV in preparation of a TAVR procedure. For example, contrast-enhanced multidetector computed tomography (MDCT) imaging is often used to acquire a series of images ranging between the neck base to the iliac arteries, where the sizing measurements of aortic annulus, aortic root, and the vessel sizes, tortuosity, and calcium distributions of the delivery system pathway are evaluated to verify the “anatomic adequacy” for a TAVR patient candidate. That is, during a TAVR procedure, accurate catheter alignment and valve deployment are the essential steps to ensure (a) proper functionality of implanted THV, (b) reduce the risk of coronary flow obstructions, (c) avoid valve migration or late embolization, and (d) minimize the risk of aortic root injury. As such, prior to replacement of the THV, the alignment of THV catheter marker against the projection of aortic annulus is determined by the use of imaging modality to evaluate the proper size of the targeted anatomy because if the implanted THV is placed too high, potential coronary flow obstructions may occur to prevent from normal blood flow from coronary arteries to heart muscles causing acute myocardial infarction (AMI). And if the THV is implanted too low relative to the aortic annulus, the prosthetic leaflets may not fully open, thereby causing a weak or instable hemodynamic flow (i.e., aortic insufficiency with high pressure gradient) or the frame of valve may be deformed resulting in complications such as paravalvular regurgitation, rhythm disturbances.

Preoperative information, such as patient selection and correct matching to a specific THV size, is, therefore, typically obtained with MDCT imaging methods for patient selection, device size selection, and the preprocedural evaluation of possible access routes. For example, conventional computed tomography (CT) images are visualized in three standard viewing angles including sagittal, coronal and axial to create a two-dimensional (2D) image based on a selected reference point. Multiplanar reformation (MPR) is the process of using the data from the original cross-sectional CT images to create non-standard two-dimensional (2D) images to better visualize the region-of-interest.

Identification of the coronary cuspid landmarks or nadirs play a key role in determining the reference points for the 2D images. The nadirs are currently identified manually upon inspection of 2D CT images based on the 3D multiplanar reformation (MPR) or the 2D planimetric technique using the pre-acquired CT images of the patient.

### SUMMARY

What is needed is a more accurate and automated way of identifying the coronary cuspid landmarks or nadirs. The proposed methods and systems of the present disclose a computer implemented technique that includes utilizing structured 3D dataset of cusps to simultaneously determine the nadirs. Such method and systems are especially beneficial, in comparison to existing techniques, when the sizes of cusps have significant difference (i.e., Type 1 aortic valve with two fused leaflets) or the aortic root has a bicuspid configuration rather than a tricuspid configuration. That is, instead of manually determining nadirs based on a 2-D planimetric method subject to intra- and inter-operator errors and inconsistency, a computational technique is proposed to automatically identify the nadirs simultaneously based on the 3D skeletonized datasets (i.e., surface points of cusps and the centerline or skeleton of the aortic root) that are derived from the CT images.

In first example, a non-transitory computer readable medium has a computer program stored thereon for performing a direct three-dimensional (3D) modeling technique for or in preparation for a transcatheter aortic valve replacement (TAVR) procedure. The computer program comprises instructions for causing one or more processors to: receive a plurality of datasets of medical images comprising coronary anatomical structures; segmenting the medical images to produce a set of 3D surface points, wherein the set of 3D surface points represents a 3D geometrical shape of the coronary anatomical structures; performing a 3D curve-based skeletonization process to transform the set of 3D surface points to a structure-based representation of the coronary anatomical structures; segmenting the structure-based representation of the coronary anatomical structures into independent 3D objects, wherein the independent 3D objects comprise at least two coronary cusps and an extended aortic root, wherein the at least two coronary cusps and the extended aortic root are each represented by a dataset of structured 3D point coordinates; characterizing each dataset of structured 3D point coordinates as a parametric surface function; performing a minimization process on the structured 3D point coordinates and the parametric surface functions to simultaneously determine 3D coordinates of nadir points associated with each of the least two coronary cusps; using the 3D coordinates of the nadir points to calculate angiographic coplanar views and an associated cusp overlap map; and deriving an optimal view map from the angiographic coplanar views and an associated cusp overlap map, the optimal view map comprising optimal viewing configurations for imaging equipment used during the TAVR procedure.

In a second example, the non-transitory computer readable medium of the first example, wherein the computer program further comprises instructions for causing one or more processors to evaluate a degree of overlap between the at least two cusps.

In a third example, the non-transitory computer readable medium of the second example, wherein the computer program further comprises instructions for causing one or more processors to use the degree of overlap between the at least two cusps to calculate the angiographic coplanar views and the associated cusp overlap map.

While multiple examples are disclosed, still other embodiments of the present disclosure will become apparent to those skilled in the art from the following detailed description, which shows and describes illustrative examples of the disclosure. Accordingly, the drawings and detailed description are to be regarded as illustrative in nature and not restrictive.

### BRIEF DESCRIPTION OF THE DRAWINGS

FIG. 1A is a series of cross-sectional images based on the computed tomography (CT) voxel dataset viewed by different cutting planes, such as a sagittal viewing plane (when lines purple (A-A) and blue (B-B) are shown), an axial viewing plane (when lines purple (A-A) and yellow (C-C) are shown) and a coronal axial viewing plane (when lines blue (B-B) and yellow (C-C) are shown).

FIG. 1B is a series of cross-sectional images based on the computed tomography (CT) voxel dataset viewed by different cutting planes, such as a sagittal viewing plane (when lines purple (A-A) and blue (B-B) are shown, an axial viewing plane (when lines purple (A-A) and yellow (C-C) are shown) and a coronal axial viewing plane (when lines blue (B-B) and yellow (C-C) are shown), as well as a cross sectional image depicting the three nadirs of each cusp and the perimeter of the annulus of the THV.

FIG. 2 is a series of cross-sectional images of the THV depicting the heights of aortic root with respect to the LCC, RCC, and NCC, as well as the heights of left and right coronary artery ostia.

FIG. 3 is a series of cross-sectional images used to calculate the cross-sectional area of the left ventricular outflow tract (LVOT).

FIG. 4 is a series of cross-sectional images of the THV depicting the size of sinus of valsalva (SOV).

FIG. 5 is a series of cross-sectional images of the THV depicting the size of the sinus tubular junction (STJ).

FIG. 6 is a predicted angiographic view for aortic valve implant illustrated by 3D surface rendering (a) 1 and translucent maximum intensity projection (a) 2 based on chosen gantry angle shown in (b).

FIG. 7A is an angiographic view of an optimal THV implant angiographic view, wherein the side of 3D disk is visualized and the projected area of the two cusps are maximum.

FIG. 7B is a simulated X-ray projection is illustrated with the C-arm angle LAO 25, CRAN 14.

FIG. 7C is a barrel view of the 3D aorta model where the calcium distribution as shown in the RCC site.

FIG. 8A illustrates a resultant volumetric dataset for aortic root and LV chamber.

FIG. 8B is a surface-based polygonal 3D model is created transformed from the voxels of CT images.

FIG. 9A is an illustration of the individual anatomical structures identified and modularized from the original 3D polygonal dataset resulting in a separate LCA model.

FIG. 9B is an illustration of the individual anatomical structures identified and modularized from the original 3D polygonal dataset resulting in three individual LCC, RCC, NCC cusp models.

FIG. 9C is an illustration of the individual anatomical structures identified and modularized from the original 3D polygonal dataset resulting in a final structured aortic model consisting of individual anatomical structures decomposed to a series of contours.

FIG. 10A is an optimal map derived based on co-planar nadirs.

FIG. 10B is an optimal map derived based on cusp overlap.

FIG. 10C is an optimal map derived based on the integration of co-planar and cusp overlap.

FIG. 11 is a summarized evaluation illustrating the implant C-arm gantry angles LAO 2 CAUD 15 (i.e., angiographic view) associated with the 3D surface-based rendering of aortic root (upper left panel), the 3D translucent rendering of aortic root for the calcifications of the aortic root (upper middle panel) and barrel viewing angles (upper right panel). The corresponding optimal view map with the star symbol (lower left panel) indicates the suggested C-arm gantry angle (i.e., LAO 2, CUAD 15), and the volumetric data sets to depict the spatial relationships between the aortic root and heart chambers (lower middle panel) and the catheter delivery pathway consisting of descending aorta and iliac arteries.

FIG. 12 is an illustration an cantilevered X-ray system used to produce the 2D angiographic images during the TAVR procedure where the C-arm gantry can be rotated with respect to patient's body from left to right as LAO and RAO angles and from head to toes as CRAN and CAUD angles.

FIG. 13 is an illustration of a block diagram of an example computer system which may be used to implement all or certain or a combination of the methods illustrated in FIG. 14 and/or implement all or certain or a combination of aspects of the examples discussed herein.

FIG. 14 is an illustration of a flow diagram of techniques to generate information as pre-procedural TAVR planning, in accordance with the present disclosure.

FIG. 15 is a schematic diagram of an aorta root including the ascending aorta (AO), sinus of Valsalva (SOV), and left ventricle outflow tract (LVOT). The VBRO is first determined from the three nadir points either calculated from the computational method or manual method where the annular size in terms of min/max diameters, perimeter, and area are automatically calculated. Other estimates are calculated by first determining the respective cross-sectional area resulting from the cutting planes Planex where x={LVOT, SOV, STJ, AO}derived from moving the VBRn plane up and down to intersect the segmented 3D aortic model. The linear distance-based estimates such as STJ heights (LCC_{H}/RCC_{H}/NCC_{H}), coronary ostial heights (LCA_{H}/RCA_{H}), and LCA/RCA leaflet length (LCA_{L}/RCA_{L}) can be determined by calculating the distance between the landmarks or onto the targeted plane.

FIG. 16A illustrates the common elliptical contour of general region-of-interests (ROIs) resulting from the intersections of different plane and the aortic root. The min/max diameter is estimated by the major/long and minor/short axial directional vectors determined by Principal Component Analysis theory.

FIG. 16B illustrates the specific contour of SOV consisting of three cusps where the three diamond symbols corresponding to the outer points of cusps at the commissure joint level and the square symbols denote the junction of the two adjacent cusps.

FIG. 17A illustrates a typical segmented 3D aortic model from a patient's CT images and 3D rendered with pink surfaces where the calculated estimates and the associated contours are mapped or superimposed to the 3D aortic model for 3D rendering.

FIG. 17B illustrates calculated estimates tabulated as a list for quick access and review for optimal determination of type and size of a THV.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

As set forth above, embodiments disclosed herein, which are rooted in computer technology (e.g., machine learning) may reduce some of the shortcomings associated with conventional systems, methods and/or models used in performing a transcatheter aortic valve replacement (TAVR) procedure.

Prior to performing a TAVR procedure, certain preoperative information is obtained. Typically, most preoperative information for patient selection and the matching of a specifically sized implantable transcatheter heart valve (THV) is obtained with contrast-enhanced multidetector computed tomography (MDCT) imaging methods in patient selection, device size selection, and the preprocedural evaluation of possible access routes. As discussed above, MDCT imaging is often used to acquire a series of images ranging between the neck base to the iliac arteries, where the sizing measurements of aortic annulus, aortic root, and the vessel sizes, tortuosity, and calcium distributions of the delivery system pathway to evaluate and verify the “anatomic adequacy” prior to commencing a TAVR procedure.

Typical computed tomography (CT) images are traditionally visualized in three standard viewing angles including axial, coronal, and sagittal plane to create a two-dimensional (2D) image based on the selected reference point. Multiplanar reformation (MPR) is the process of using the data from the original cross-sectional CT images to create non-standard two-dimensional (2D) images to best visualize the region-of-interest. Again, one area of interest is the aortic annulus, and the process of evaluating this annular region is to determine the virtual basial plane that pass through the nadirs of three cusps or two cusps (i.e., in a bicuspid aortic anatomy).

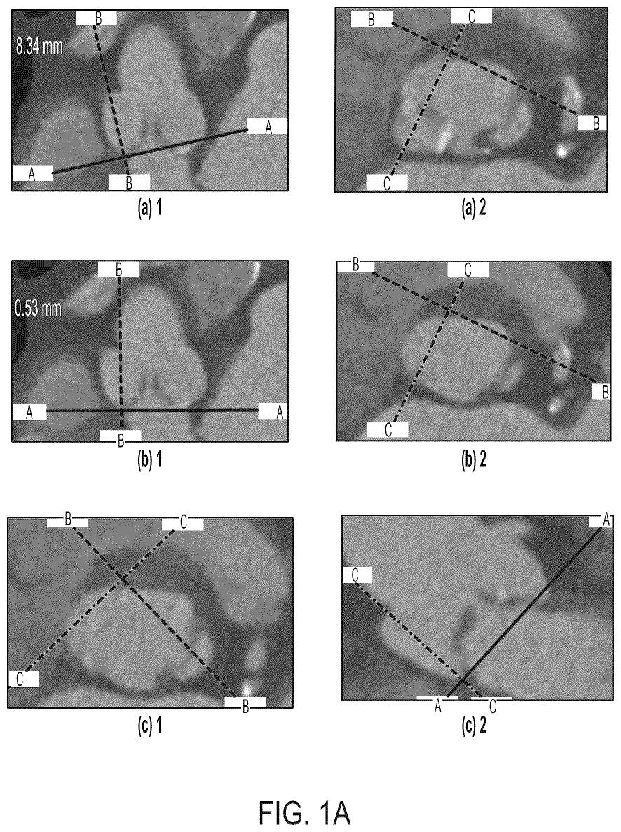

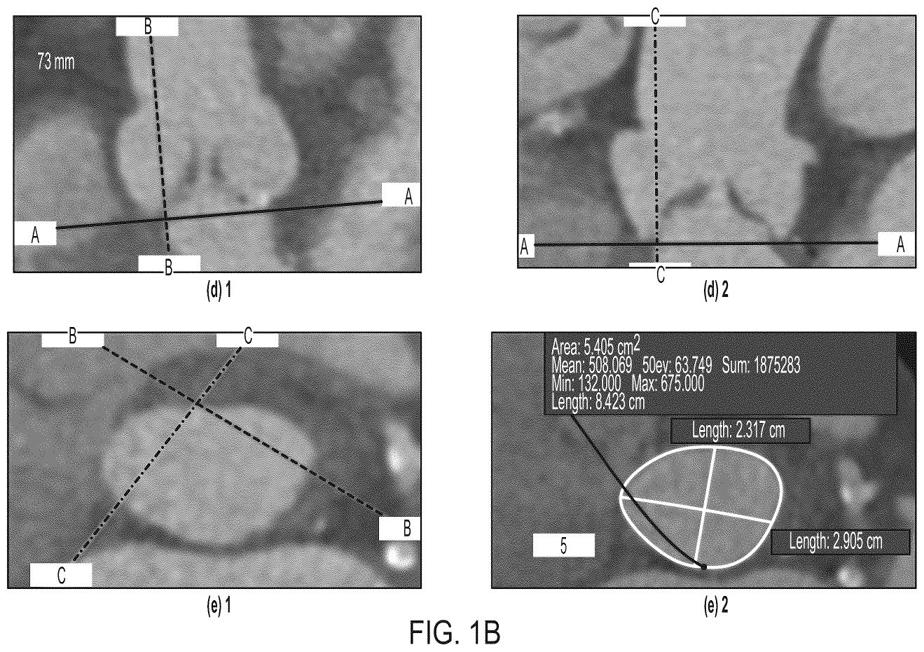

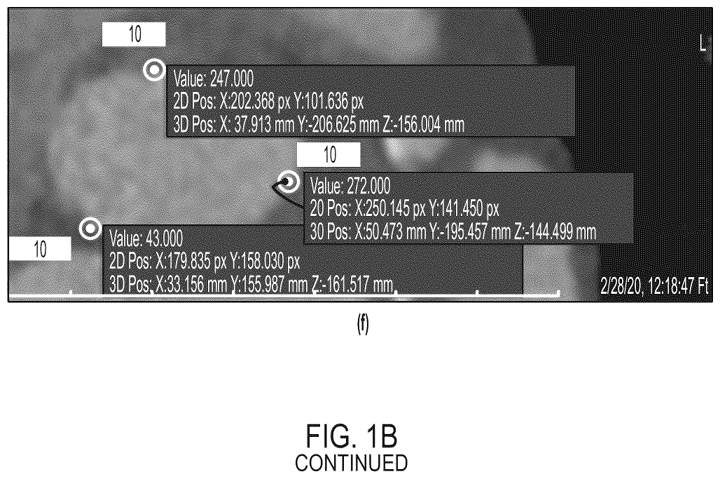

Referring to FIGS. 1A and 1B, there are depicted a series of cross-sectional images based on the CT voxel dataset(s) viewed by different cutting planes used to identify the three nadirs of each cusp and the perimeter of the aortic annulus of the THV. For example, images identified as (a)1 and (b)1 of FIG. 1A are sagittal viewing planes because lines purple (A-A) and blue (B-B) are shown, image identified as (c)2 of FIG. 1A is a coronal viewing plane because lines purple (A-A) and yellow (C-C) are shown, and image identified as (a)2, (b)2 and (c)1 of FIG. 1A are axial viewing planes because lines blue (B-B) and yellow (C-C) are shown. Unless otherwise discussed herein, the sagittal viewing plane is depicted when lines purple (A-A) and blue (B-B) are shown in the 2D image, the coronal viewing plane is depicted when lines purple (A-A) and yellow (C-C) are shown in the 2D image, and the axial viewing planes is depicted when lines blue (B-B) and yellow (C-C) are shown in the 2D image. These images are used to identify and depict the three nadirs of each cusp and the perimeter of the aortic annulus of the THV.

The first step to calculate the perimeter of the aortic annulus of the THV is to determine any one nadir (e.g., right coronary cusp or RCC) among the three cusps. Referring to images (a)1 and (a)2 of FIG. 1A, the sagittal plane image (a)1 is moved forward and backward, and the axial plane image (a) 2 is moved up and down to identify the lowest location of the targeted cusp in the form of the intersection between the blue line (B-B) and purple line (A-A), as shown in image in image (a)1. Once the first nadir is determined, the orange line (C-C) is rotated to pass through the first nadir and the centroid of the second cusp based on the axial viewing plane (e.g., non-coronary cusp or NCC) as shown in image (b)1. Once the position of the orange line is determined, the reference axis (purple line (A-A)) on the sagittal viewing plane is rotated as shown in image (b)2 to identify the nadir of the second cusp until its shape is totally gone as shown in image (b)2. Once the first and second nadirs are determined, the third nadir (e.g., left coronary cusp or LCC) is determined by rotating the reference axis (i.e., blue line (B-B)) on axial viewing plane such that it passes through the centroid of the third cusp as shown on the left panel in image (c)1. By rotating the reference axis (i.e., purple line (A-A)) as shown in image (c)2, the nadir can be determined when the third cusp's bottom vanishes as shown in image (c)2 and all three nadirs are aligned right on the top of reference line (purple line (A-A)) as shown in the images (d)1 and d(2) in FIG. 1B. The resultant three nadirs define a unique plane that serves as the desired virtual basal plane. The virtual basal 2D plane is depicted as image (e)1 of FIG. 1B. The 2D contour and long and short segments associated with the aortic annulus (e.g., annular region) are manually drawn where the perimeter and area of the 2D contour are calculated mathematically as shown in image (e)2 of FIG. 1B. That is, image (e)2 of FIG. 1B depicts the contour of the aortic annulus, as well as the minimum and maximum diameters of aortic annulus, thereby yielding the perimeter and area estimates of the aortic annulus. And image (f) of FIG. 1B depicts the three nadirs—LCC, RCC, and NCC—as well as their respective 2D and 3D coordinates/positions.

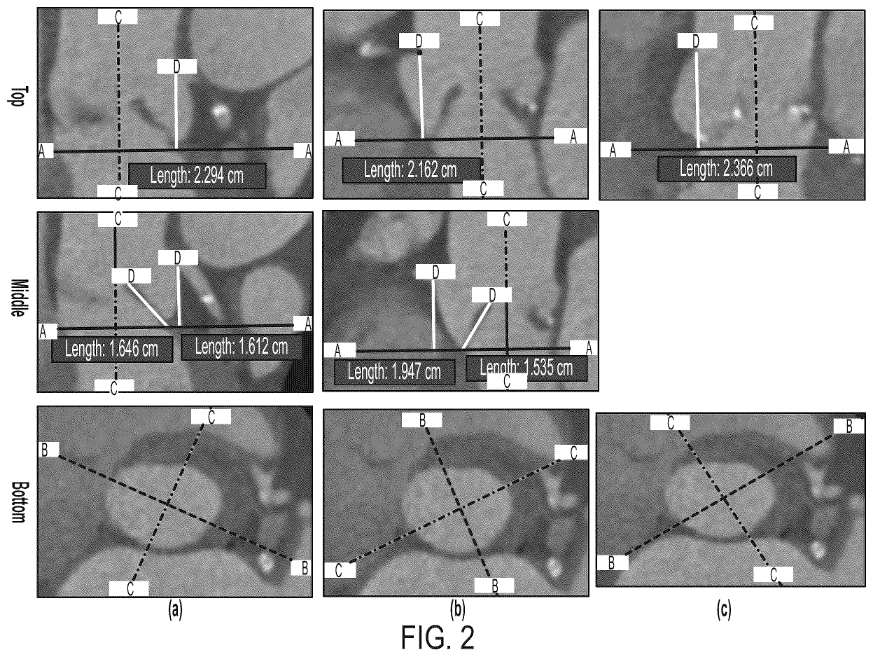

Referring to FIG. 2, there is depicted a series of cross-sectional images of the THV for determining and calculating the heights of aortic root with respect to the LCC, RCC, and NCC, as well as the heights of left and right coronary artery ostia. Now that the LCC, RCC, and NCC coordinates are determined based on images (e)1, (e)2 and (f) of FIG. 1B shown in the virtual basal plane, the heights of the aortic root with respect to the LCC, RCC, and NCC, as well as the heights of the left and right coronary artery ostia can be calculated. Based on the defined virtual basal plane, the blue line (B-B) in image (e)1 of FIG. 1B—which is the same the blue line (B-B) in image (a) Bottom of FIG. 2—depicts the axial viewing plane, and the blue line (B-B) is rotated such that it passes through the nadir of the LCC (i.e., 4 o'clock direction) to calculate the height of the LCC (Top Left) and LCA ostial height and leaflet (Middle Left). Based on the defined virtual basal plane, the blue line (B-B) in image (b) Bottom of FIG. 2 is rotated such that it passes through the nadir of the RCC (i.e., 11 o'clock direction) to calculate the height of the RCC (Top Center) and NCC (Top Right) and RCA ostial height and leaflet (Middle Right). Based on the defined virtual basal plane, the blue line (B-B) in image (c) Bottom of FIG. 2 is rotated such that it passes through the nadir of the NCC (i.e., 2 o'clock direction) to estimate the height from the annular plane to the sinus tubular junction (STJ).

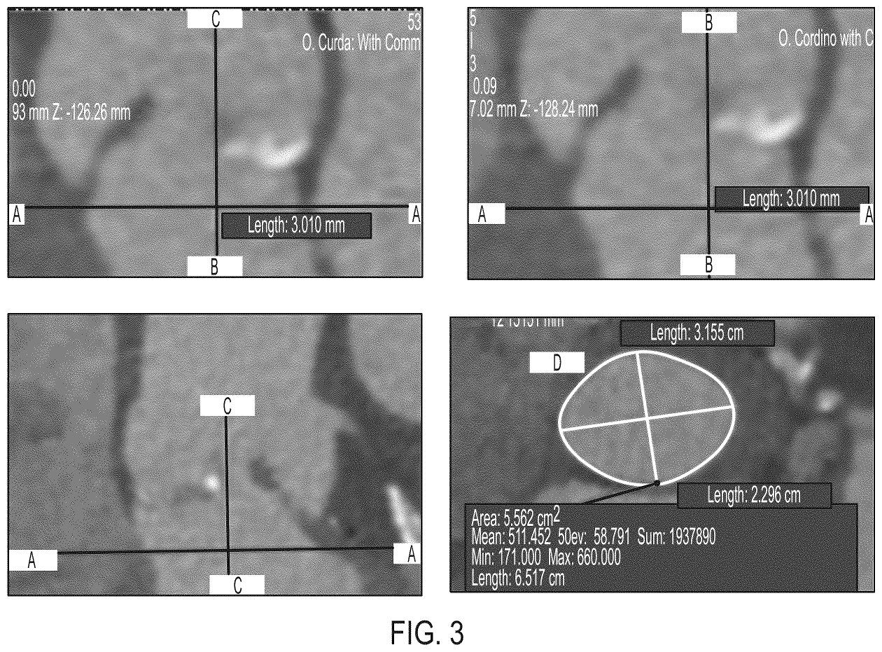

Referring to FIG. 3, there are depicted a series of cross-sectional images used to calculate the cross-sectional area of the left ventricular outflow tract (LVOT). Referring to image (a) of FIG. 3, which depicts the virtual basal plane, a reference segment is defined where its proximal end is perpendicularly placed under the virtual basal plane (purple line (A-A)). Referring to image (b), the virtual basal plane is moved downward until it is descended to the desired distance (e.g., 3 or 4 mm below the virtual basal plane). Referring to image (c), the new position of virtual plane now serves as the LVOT plane. And the cross-sectional diameters, perimeter, and area of the LVOT are evaluated (e.g., manually defined and calculated) after the boundary of the cross-sectional area of the LVOT is drawn, as depicted in FIG. 3.

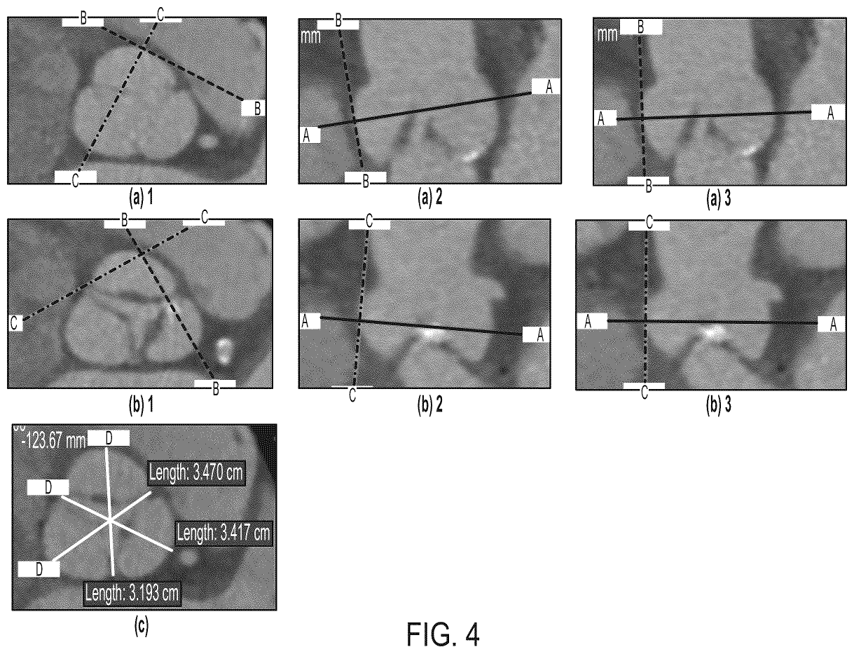

Referring to FIG. 4, there are depicted a series of cross-sectional images of the THV used to calculate the size of sinus of Valsalva (SOV). The size of the SOV is evaluated by first determining the plane intersecting the aortic root and being perpendicular to the long axis of aortic root such that the perimeter of the cross-sectional boundary is at a maximum followed by manually defining the individual three segments connecting from the outmost boundary point at one cusp toward the commissure or joint point formed by the other two cusps. Referring to image (a)1 of FIG. 4, the orange line (C-C) is rotated on the axial view plane such that it passes the top point of the RCC (i.e., intersection point of blue line (B-B) and orange lines (C-C)) and the centroid of the NCC (i.e., 7 o'clock direction) yielding the cross-sectional image on the sagittal viewing plane as shown in image (a)2. Referring to image (a)3, by rotating the purple line on the sagittal viewing plane to intersect the NCC at the middle curve (i.e., at the 3 o'clock direction), the current SOV plane cuts through the largest diameters of the RCC and NCC. The blue line (B-B) is rotated to intersect the middle curve of the LCC on the axial viewing plane as shown in image (b)1 followed by rotating the purple line (A-A) as shown in image (b)2 to intersect the middle point of the LCC curve as shown in image (b)3. Referring to image (c), based on the resultant SOV plane passing through the largest cross-sectional contour of cusps, each line segment connecting from the farthest point at the contour of one cusp with respect to the centroid of SOV to the commission point between the other two adjacent cusps is drawn as the largest cross-sectional distance associated with the current cusp.

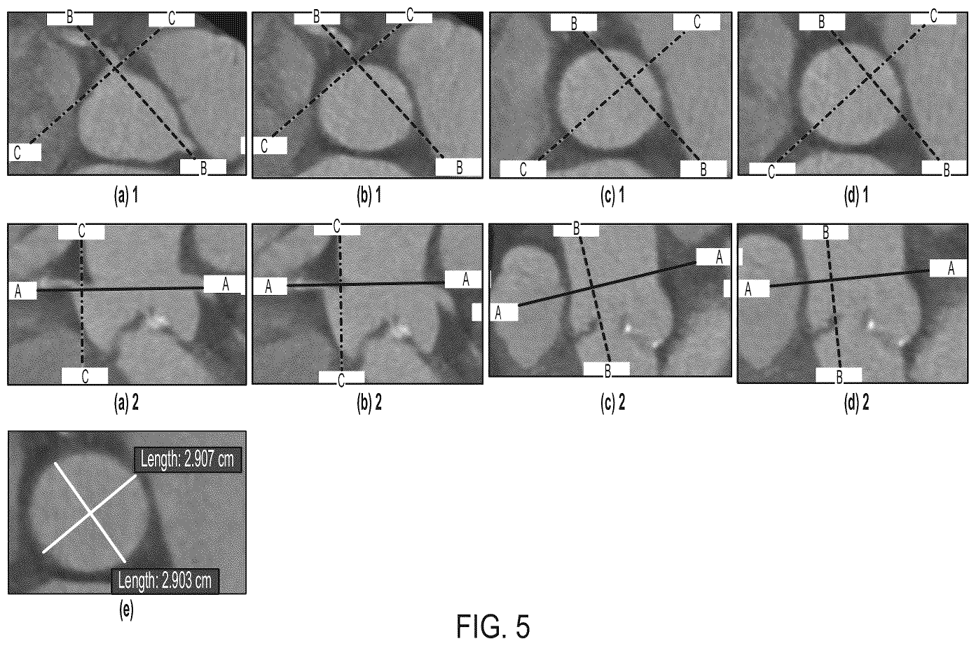

Referring to FIG. 5, there are depicted a series of cross-sectional images of the THV used to calculate the size of the sinus tubular junction (STJ). The size of the STJ is evaluated by first defining the plane passing through the upper locations of LCA and RCA ostia and the top location of the NCC followed by manually determining the long and short segments based on the boundary of the STJ. With the STJ plane determined, another plane is defined to evaluate the size of the ascending aorta in terms of long and short axes based on the boundary of the cross-sectional area. Referring to images (a)1 and (a)2, the intersection point of orange line (C-C) and blue line (B-B) is placed at the RCA ostium such that the blue line (B-B) passes through the LCA ostium as shown on the top images on the axial viewing plane yielding the cross-sectional image with sagittal viewing plane cuts through the two coronary ostia as shown on the bottom images. Referring to images (b)1 and (b)2, the sagittal viewing plane in image (b)2 is moved up to passing through the STJ region as shown on the bottom panel yielding new cross-sectional image on the axial viewing plane as illustrated in image (b)1. Referring to images (c)1 and (c)2, the orange line (C-C) is shifted to the center of ascending aorta as shown in image (c)1 of the axial viewing plane yielding the cross-sectional image on the sagittal viewing plane that passes through a higher location at the STJ above the NCC as shown in image (c)2. Referring to images (d)1 and (d)2, by rotating the purple line (A-A) on the sagittal view plane in image (d)2, it passes through the exact location of the STJ at the NCC resulting in the cross-sectional image at the STJ level as shown in image (d)1. Based on the size of the STJ in term of minimum and maximum diameters are drawn as shown in image (e) of FIG. 5, and the respective distances are calculated.

The previous paragraphs discussed herein disclose the preoperative steps for a TAVR, which includes replacing the patient's original aortic valve that fails to open properly with an implantable, bioprosthetic THV. Transcatheter valve-in-valve (ViV), alternatively, involves replacing the failing implanted, bioprosthetic valve with a new implantable, bioprosthetic THV. Although the size of the previously, implanted, bioprosthetic valve may be known, its actual shape may have become deformed under cardiac motion. Additionally, pannus overgrowth or in-valve calcification deposits or thrombosis may have altered the size of the annular region. Similar to preparing for a TAVR procedure, it may be desirable to optimize the type, size, and implant position of the THV used in a ViV procedure based on an individual patient's anatomy. As such, the same or similar steps in preparing for and performing a TAVR procedure are also applicable in preparing for and performing a ViV procedure. That is, when performing a ViV procedure, the nadirs of the previously, implanted, bioprosthetic valve may be identified by using similar approaches as adopted for defining the nadirs of native aortic valve.

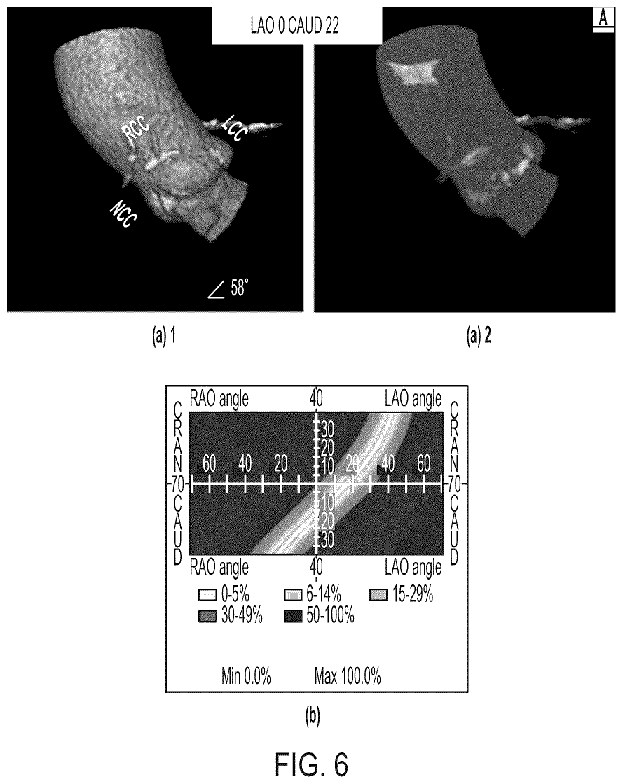

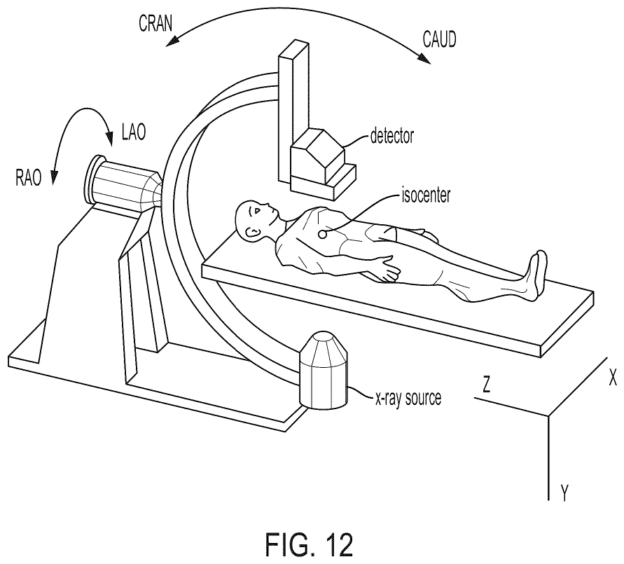

As discussed above, to achieve a successful TAVR procedure, it is important to position and deploy the THV accurately. This can be achieved by using a fluoroscopic view perpendicular to the native valve, sometimes called the “coplanar” view as discussed above. The 3D coordinates of each nadir manually defined above are used to calculate the angiographic coplanar views resulting in a coplanar curve map as shown in image (b) of FIG. 6. The coplanar curve, as shown on the map illustrated in image (b) of FIG. 6, defines the relationship between the C-arm gantry angulation (i.e., LAO 60 to RAO 60 and CRAN 45 to CAUD 45) and the projections of the nadirs. Referring to FIG. 12, there is depicted a cantilevered X-ray system used to produce the cross-sectional images based on CT. The angulation system uses the following defined terminology to calculate the C-arm's position and angles: RAO means the image intensifier/detector/camera resides on the right of the patient; LAO means the image intensifier/detector/camera resides on the left of the patient; CRAN means Cranial and towards the patient's head; and CAUD means Caudal and towards the patient's feet. Referring again to FIG. 6, image (b) the white curve of the co-planar map denotes the multiple solutions of the C-arm gantry trajectory (i.e., various angulations) where the nadirs form a line on the projection based on the chosen gantry angle.

3D rendering images of the segmented aortic root are displayed by use of the predicted C-arm gantry angles associated with the coplanar curve in order to visually select the best angiographic view where the projected nadirs lay on a line and the three cusps are symmetrically separated among each other on the projection view. Referring to image (a)1 of FIG. 6, the predicted angiographic view for aortic valve implant illustrated by 3D surface rendering is shown and referring to image (a)2 of FIG. 6, the translucent maximum intensity projection is shown.

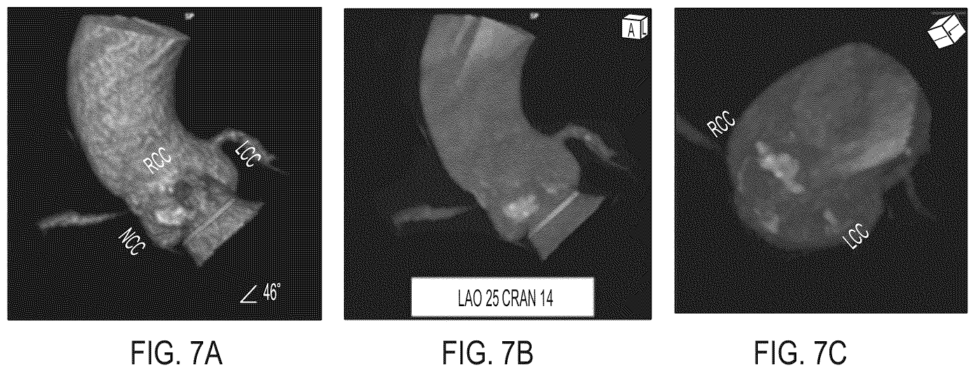

FIGS. 7A, 7B and 7C illustrate images facilitating determination of annulus for a bicuspid valve. More specifically, FIG. 7A illustrates an optimal THV implant angiographic view, wherein the side of 3D disk is visualized and the projected area of the two cusps are maximum. FIG. 7B is a simulated X-ray projection with a C-arm angle of LAO 25, CRAN 14. FIG. 7C is a barrel view of the 3D aorta model where the calcium distribution are shown in the RCC site.

Rather than identifying the nadirs manually or based on planimetric MPR method or calculating individual nadir based on computational methods, as discussed above, the methods and systems of the present disclosure utilize structured 3D cusp datasets to simultaneously determine the nadirs, which yield more accurate results than the existing techniques. The methods and systems of the present disclosure utilizing structured 3D cusp datasets to simultaneously determine the nadirs are especially beneficial, in comparison to existing techniques, when the sizes of cusps have significant difference (i.e., Type 1 aortic valve with two fused leaflets) or the aortic root has a bicuspid configuration rather than a tricuspid configuration.

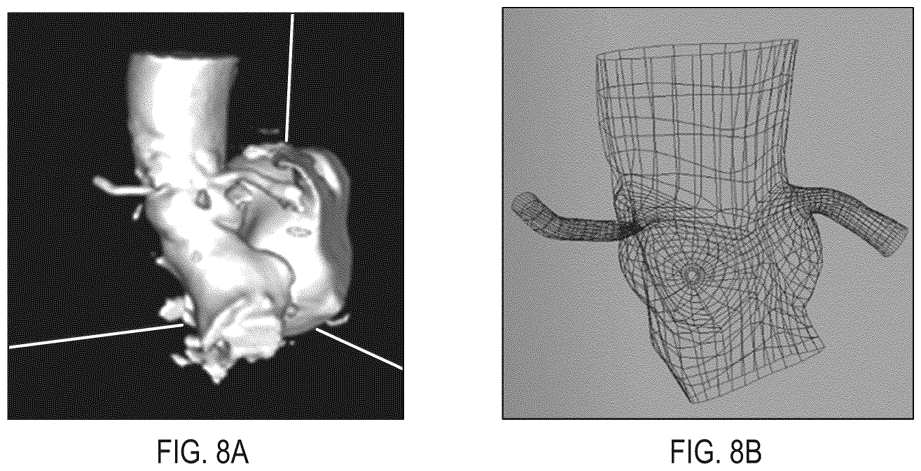

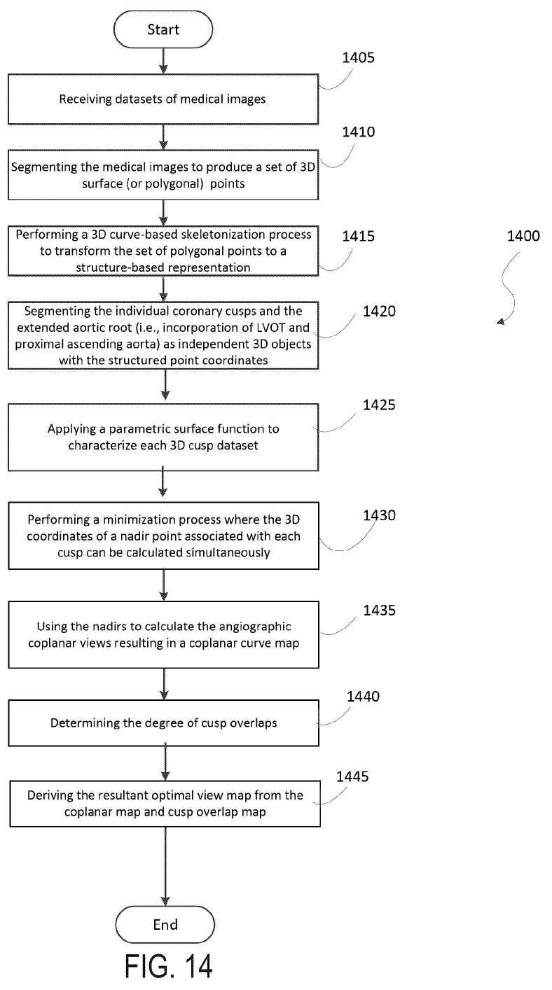

Referring to FIG. 14, there is depicted a flow diagram for a method 1400 of performing a computational based 3D modeling technique and analysis for assisting a TAVR procedure, in accordance with the present disclosure. Step 1405 includes receiving datasets of medical images of coronary anatomical structures of the patient, such as CT, magnetic resonance (MR) or 3D TEE images. Receiving the datasets of medical images may include importing the images via a picture archiving and communication system (PACS). Step 1410 includes segmenting the medical images to produce a set of 3D surface points, which represent the 3D geometrical shape of associated anatomical structures, as shown in FIG. 8B. Segmenting the medical images may include one or more user operations to “remove” or “include” voxels of the dataset to form multiple meaningful entities corresponding to relevant anatomical structures, such as the aortic root, chambers, and/or blood vessels. By using a combined region-growing technique and voxel cutting-and-pasting process, the relevant anatomical structures corresponding to calcifications and the aortic root are formed first followed by a multi-thresholding method to convert the segmented voxels to different datasets comprising 3D coordinate points (i.e., STL or OBJ format) for individual anatomical components.

Step 1415 includes performing a 3D curve-based skeletonization process to transform the set of polygonal points generated from Step 1410 to a structure-based representation in terms of a 3D skeleton or median curve (i.e., approximated by a series of points) associated with a set of contours perpendicular to the points of the median curve. The 3D curve-based skeletonization process may include using one or a combination of commercially available software packages. Such software packages may include products sold under the tradenames OsIriX™, Modo™, MeshLab™ Rhino™ and Excel™. OsiriX is an image processing application for Mac dedicated to DICOM images produced by equipment. OsiriX is complementary to existing viewers, in particular to nuclear medicine viewers; it can also read many other file formats: TIFF, JPEG, PDF, AVI, MPEG and QuickTime. Modo is a polygon and subdivision surface modeling, sculpting, 3D painting, animation and rendering package developed by Luxology, LLC, which is now merged with and known as Foundry. The program incorporates features such as n-gons and edge weighting, and runs on Microsoft Windows, Linux and macOS platforms. MeshLab is a 3D mesh processing software system that is oriented to the management and processing of unstructured large meshes and provides a set of tools for editing, cleaning, healing, inspecting, rendering, and converting these kinds of meshes. Rhino is a 3D modeler used to create, edit, analyze, document, render, animate, and translate NURBS* curves, surfaces, and solids, point clouds, and polygon meshes. Excel is a spreadsheet.

For example, the combination of software packages may be used as follows: (a) use Osirix to perform quick volume editing and export aortic root region as an .obj file; (b) use Modo to import and quickly cleanup the .obj file from Osirix, generate an UV map, re-export for curvature analysis, and create layer structure for contour skeleton creation; (c) use MeshLab to import the cleaned aortic root file from Modo, create a surface curvature map, and save the surface curvature map; use Modo to apply curvature map from MeshLab, create a contour skeleton for the root and cusps, export new .obj file; (d) use Rhino to import the contour skeleton structure from Modo, extract curves from objects, extract points from curves, export points as text files; and use Excel to use a template file to modify text output from Rhino into a proper contour object format.

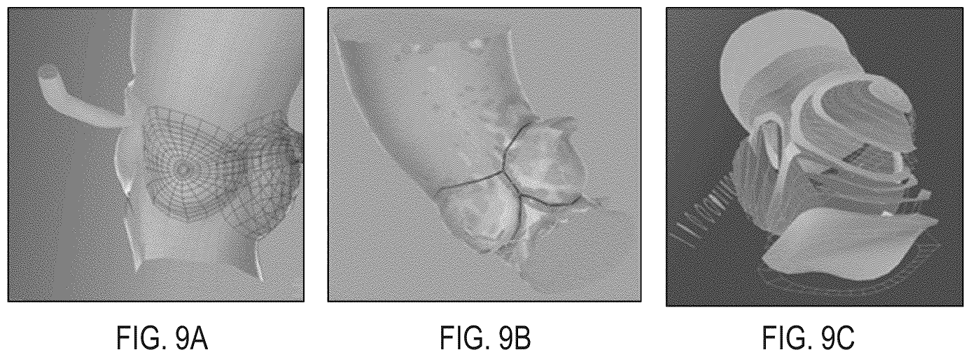

The transformed 3D skeletonized datasets resulting from Step 1415 will be in the form of a series contours having (1) coronary cusps (i.e., LCC, NCC, and RCC) with the respective proximal LCA and RCA segments extended from the LCC and RCC, (2) main aortic anatomy including the LVOT, aortic root, SOV, STJ, and proximal ascending aorta, and (3) calcified blocks, which are subsequently used for quantitative analyses. Referring to FIG. 9A, there is depicted an illustration of the individual anatomical structures identified and modularized as the set of polygonal points from the original 3D polygonal dataset. Referring to FIG. 9B, there is depicted an illustration of the individual anatomical structures identified in FIG. 9A, resulting in the individual sub-datasets corresponding to the LCC, RCC, and NCC cusps. Referring to FIG. 9C, the individual sub-datasets consisting of polygonal surface points are further transformed to its final structured model in the form of a series of contours.

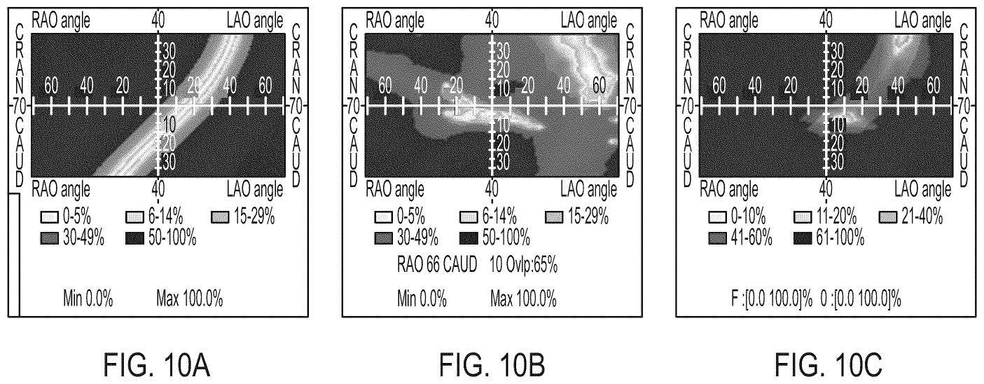

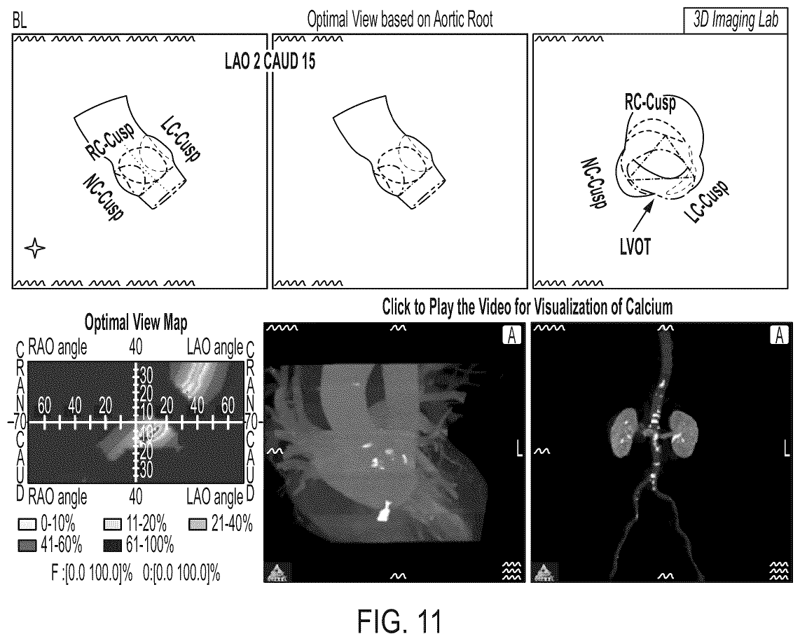

Instead of manually and serially determining the nadirs of the THV based on a 2D planimetric method, the present disclosure discusses a computational technique that automatically identifies the nadirs simultaneously based on the 3D skeletonized datasets discussed above, thereby facilitating calculation of a virtual basal/annular plane and angiographic view for the implantation of the THV. For a tricuspid aortic root, the individual coronary cusps and the extended aortic root (i.e., incorporation of the LVOT and proximal ascending aorta) are segmented as independent 3D objects with the structured point coordinates denoted as P^{LCC}, P^{RCC}, P^{NCC} and P^{AO} respectively, as shown in Step 1420. Based on the structured 3D points, a parametric surface function is employed to characterize each 3D cusp dataset as Sⁱ(u,v), where symbol i denotes one of the LCC, RCC, NCC, or extended aortic root, and u, v denote the parametric variables of the corresponding parametric surface function, as shown in Step 1425. Based on the directional vector V^{AO} derived from the 3D dataset P^{AO}, and the three parametric surface function Sⁱ(u,v), a minimization process is performed where the 3D coordinates of a nadir point associated with each cusp can be calculated simultaneously, as shown in Step 1430. Step 1435 includes using the nadirs to calculate the angiographic coplanar views resulting in a coplanar curve map. Step 1440 includes evaluating the degree of cusp overlap. Step 1445 includes using the information derived from the respective coplanar map and cusp overlap map, the resultant optimal view map can be derived where the contents on the integrated maps define a projection view in terms of C-arm gantry angulation (i.e., LAO 60 to RAO 60 and CRAN 45 to CAUD 45) with the associated coplanar and cusp overlap estimates. Alternatively, the cusp overlap map is not used, and instead a 3D rendering of aorta root (for example, as shown in FIG. 8A or FIG. 9A) may be used with the coplanar map (for example, as shown in FIG. 10A) to determine cusp overlap visually by rotating the 3D model with the corresponding C-arm gantry angles (for example LAO 10 CRAN 20).

Regarding Step 1430, the minimization process can include the use of the following equations. Let P^{LCC}, P^{RCC}, P^{NCC} and P^{AOE} denote the individual coronary cusps and the extended aortic root (i.e., incorporation of the LVOT and proximal ascending aorta) that are segmented 3D objects respectively. Based on the structured 3D points, a parametric surface function is employed to characterize each 3D cusp dataset as Sⁱ(u,v), where symbol i denotes one of the LCC, RCC, NCC, or extended aortic root AOE, and u, v denote the parametric variables of the corresponding parametric surface function for each cusp. A minimization process is employed to calculate the three sets of parametric variables that defines the resultant 3D coordinates of the nadirs by use of the following equations where the 3D coordinates of nadir points associated with each cusp can be calculated simultaneously.

$$min ℱ ( [ \begin{matrix}{u}^{Lcusp} \\ {v}^{Lcusp}\end{matrix} ] , [ \begin{matrix}{u}^{Rcusp} \\ {v}^{Rcusp}\end{matrix} ] , [ \begin{matrix}{u}^{Ncusp} \\ {v}^{Ncusp}\end{matrix} ] ) = {\sum }_{j = 1}^{n} {f}^{Lcusp} + {\sum }_{j = 1}^{m} {f}^{Lcusp} + {\sum }_{j = 1}^{k} {f}^{Lcusp}$$

$${\sum }_{j = 1}^{n} {f}^{L c u s p} = {\sum }_{j = 1}^{n} a \times {P}_{j}^{L C C} ( x ) + b \times {P}_{j}^{L C C} ( y ) + c \times {P}_{j}^{L C C} ( z ) + d$$

$${\sum }_{j = 1}^{m} {f}^{L c u s p} = {\sum }_{j = 1}^{m} a \times {P}_{j}^{R C C} ( x ) + b \times {P}_{j}^{R C C} ( y ) + c \times {P}_{j}^{R C C} ( z ) + d$$

$${\sum }_{j = 1}^{k} {f}^{L c u s p} = {\sum }_{j = 1}^{k} a \times {P}_{j}^{N C C} ( x ) + b \times {P}_{j}^{N C C} ( y ) + c \times {P}_{j}^{N C C} ( z ) + d$$

where P^{LCC}, P^{RCC}, P^{NCC} denote the respective structured 3D points associated with left cusp, right cusp, and non-coronary cusp, and (a, b, c, d) denotes the 3D coplanar equation ax+by+cz+d=0 characterized by the three nadirs in terms of the individual sets of parametric variables

$$[ \begin{matrix}{u}^{Lcusp} \\ {v}^{Lcusp}\end{matrix} ] , [ \begin{matrix}{u}^{Rcusp} \\ {v}^{Rcusp}\end{matrix} ] , [ \begin{matrix}{u}^{Ncusp} \\ {v}^{Ncusp}\end{matrix} ]$$

defined as follows.

$${\vec{V}}^{Co - plane} [ \begin{matrix}{P}^{{N}_{CP}} ( x ) \\ {P}^{{N}_{CP}} ( y ) \\ {P}^{{N}_{CP}} ( z )\end{matrix} ] = [ \begin{matrix}a \\ b \\ c\end{matrix} ] = {\vec{V}}^{LCC - RCC} ⊗ {\vec{V}}^{NCC - RCC}$$

$$d = - ( a \times {P}^{{C}_{CP}} ( x ) + b \times {P}^{{C}_{CP}} ( y ) + c \times {P}^{{C}_{CP}} ( z ) )$$

$${P}^{{C}_{CP}} = [ \begin{matrix}{P}^{{C}_{CP}} ( x ) \\ {P}^{{C}_{CP}} ( y ) \\ {P}^{{C}_{CP}} ( z )\end{matrix} ] = [ \begin{matrix}\begin{matrix}\frac{{S}^{LCC} ( {u}^{Lcup} , {v}^{Lcusp} ) ( x ) + {S}^{RCC} ( {u}^{Rcup} , {v}^{Rcusp} ) ( x ) + {S}^{NOC} ( {u}^{Ncup} , {v}^{Ncusp} ) ( x )}{3} \\ \frac{{S}^{LCC} ( {u}^{Lcup} , {v}^{Lcusp} ) ( y ) + {S}^{RCC} ( {u}^{Rcup} , {v}^{Rcusp} ) ( y ) + {S}^{NOC} ( {u}^{Ncup} , {v}^{Ncusp} ) ( y )}{3}\end{matrix} \\ \frac{{S}^{LCC} ( {u}^{Lcup} , {v}^{Lcusp} ) ( z ) + {S}^{RCC} ( {u}^{Rcup} , {v}^{Rcusp} ) ( z ) + {S}^{NOC} ( {u}^{Ncup} , {v}^{Ncusp} ) ( z )}{3}\end{matrix} ]$$

$${\vec{V}}^{LCC - RCC} [ \begin{matrix}{P}^{L - R} ( x ) \\ {P}^{L - R} ( y ) \\ {P}^{L - R} ( z )\end{matrix} ] = [ \begin{matrix}{S}^{LCC} ( {u}^{Lcup} , {v}^{Lcusp} ) ( x ) - {S}^{RCC} ( {u}^{Rcup} , {v}^{Rcusp} ) ( x ) \\ {S}^{LCC} ( {u}^{Lcup} , {v}^{Lcusp} ) ( y ) - {S}^{RCC} ( {u}^{Rcup} , {v}^{Rcusp} ) ( y ) \\ {S}^{LCC} ( {u}^{Lcup} , {v}^{Lcusp} ) ( z ) - {S}^{RCC} ( {u}^{Rcup} , {v}^{Rcusp} ) ( z )\end{matrix} ]$$

$${\vec{V}}^{NCC - RCC} [ \begin{matrix}{P}^{N - R} ( x ) \\ {P}^{N - R} ( y ) \\ {P}^{N - R} ( z )\end{matrix} ] = [ \begin{matrix}{S}^{NCC} ( {u}^{Ncup} , {v}^{Ncusp} ) ( x ) - {S}^{RCC} ( {u}^{Rcup} , {v}^{Rcusp} ) ( x ) \\ {S}^{NCC} ( {u}^{Ncup} , {v}^{Ncusp} ) ( y ) - {S}^{RCC} ( {u}^{Rcup} , {v}^{Rcusp} ) ( y ) \\ {S}^{NCC} ( {u}^{Ncup} , {v}^{Ncusp} ) ( z ) - {S}^{RCC} ( {u}^{Rcup} , {v}^{Rcusp} ) ( z )\end{matrix} ]$$

where (a, b, c) is the unit vector corresponding to the normal vector of co-plane \vec{V}^{Co-plane}, and d denotes the offset distance with respect to the co-plane, \vec{V}^{LCC-RCC}, \vec{V}^{NCC-RCC} denote two vectors spanned by the nadirs with the RCC nadir S^{RCC}(u^{Rcup}, v^{Rcusp}) as the origin, Θ denotes the vectors cross product operator, and P^{C}^{_{CP}} denotes the centroid of the nadirs.

Regarding Step 1435, which includes creating a coplanar curve map based on the direct projections of the nadirs, the coronary cuspid landmarks or nadirs play a key role because the nadirs define a line or plane resulting from the projections of the nadirs on the X-ray cine angiograms. The plane is utilized as reference where the device delivery catheter comes across and the marker of THV is superimposed and aligned. Mathematically, any three points in 3D space can uniquely determine a plane. When the nadirs of 3D plane are projected onto the image plane based on a specific C-arm angulation, the results can be a point (i.e., 3 overlapping points), a 2D triangle, or a 2D line segment among which the set of projection line segments associated with different C-arm angles are the focus of interest yielding a curve on the C-arm angulation coordinate system.

The mechanical configuration of C-arm is characterized by two degrees of freedom in term of LAO-RAO and CRAN-CAUD angles. If the C-arm is placed at the head of the patient's table-bed (i.e., head-side where the C-arm plane is parallel to the long axis of bed), the rotational plane of CRAN-CAUD angulation is defined after the rotational plane of LAO-RAO angulation angles. On the contrary, if the C-arm is placed on either side of the table bed (i.e., physician-side where the C-arm plane is perpendicular to the long axis of bed), the rotational plane of LAO-RAO angulation angles is defined after the rotational plane of CRAN-CAUD angulation. Mathematically, the resultant orientations of X-ray image planes are not identical, especially if the C-arm angulation involves a large LAO-RAO or/and CRAN-CAUD angles.

In conventional software packages, computation of co-planar views based on the nadirs can only define one of the configurations as described. By use of the “Simulated Angiographic Projection” technique, the co-planar map can be easily derived based on the required C-arm set-up. Additionally, conventional techniques for computation of co-planar curve need to rely on the three nadirs or tri-leaflets. When the aortic root is a bicuspid (i.e., two nadirs), existing methods become unusable.

The following equations can be used to create the coplanar map based on the direct projection of the nadirs. For example, let T^{H}(α, β) and T^{P}(α, β) denote the transformation matrices associated with head-side and physician-side of C-arm configuration, respectively where α and β denote the LAO-RAO, and CRAN-CAUD angles of the C-arm gantry. Stated another way, T^{H}(α, β) is associated with a configuration of the C-arm on the short end of the patient's table-bed and T^{P}(α, β) is associated with a configuration of the C-arm on the long end of the patient's table-bed. Let Nᵢ^{3d}=(Pₓⁱ, P_{y}ⁱ, P_{z}ⁱ), denote the 3D coordinates of nadir points where i=1,2,3 corresponding to the identified nadir of each cusp. Based on the transformation matrix, the 2D projection point of each nadir Nᵢ^{2d}, can be calculated as follows.

$${N}_{i}^{2 d} = ( {\overline{P}}_{x}^{i} , {\overline{P}}_{y}^{i} ) = ( {\hat{P}}_{x}^{i} / {\hat{P}}_{z}^{i} , {\hat{P}}_{y}^{i} / {\hat{P}}_{z}^{i} )$$

$$[ \begin{matrix}{\hat{P}}_{x}^{i} \\ {\hat{P}}_{y}^{i} \\ {\hat{P}}_{z}^{i}\end{matrix} ] = {T}^{H} ( α , β ) \cdot [ \begin{matrix}{P}_{x}^{i} \\ {P}_{y}^{i} \\ {P}_{z}^{i}\end{matrix} ] or [ \begin{matrix}{\hat{P}}_{x}^{i} \\ {\hat{P}}_{y}^{i} \\ {\hat{P}}_{z}^{i}\end{matrix} ] = {T}^{P} ( α , β ) \cdot [ \begin{matrix}{P}_{x}^{i} \\ {P}_{y}^{i} \\ {P}_{z}^{i}\end{matrix} ]$$

$$where$$

$${T}^{H} ( α , β ) = [ \begin{matrix}cos β & sin α sin β & - cos α sin β \\ 0 & cos α & sin α \\ sin β & - sin α cos β & cos α cos β\end{matrix} ]$$

$${T}^{P} ( α , β ) = [ \begin{matrix}cos β & 0 & - sin β \\ sin α sin β & cos α & sin α cos β \\ cos α sin β & - sin α & cos α cos β\end{matrix} ]$$

By use of the projected 2D nadir coordinates Nᵢ^{2d}=(P̅ₓⁱ, P̅_{y}ⁱ) the linearity of three nadir points £(α,β) on the image plane can be defined given the α and β corresponding to the C-arm angles as follows.

$${ℒ}^{3} ( α , β ) = \vec{{v}_{1}} ⊗ \vec{{v}_{2}} , \vec{{v}_{1}} = ( {\overline{P}}_{x}^{1} - {\overline{P}}_{x}^{0} , {\overline{P}}_{y}^{1} - {\overline{P}}_{y}^{0} ) , \vec{{v}_{2}} = ( {\overline{P}}_{x}^{2} - {\overline{P}}_{x}^{0} , {\overline{P}}_{y}^{2} - {\overline{P}}_{y}^{0} ) ,$$

where “Θ” denotes the cross product operator. When the three projected nadirs on the image plane lie in a line segment based on the given C-arm angulation (α, β), the £(α, β) value will be close to zero. Due to the digitization error, an angiographic view is regarded as co-planar view based on C-arm angulation (α, β) if the calculated £(α, β)<ϵ, where ϵ denotes a small fraction number (e.g., 10⁻²).

If a bicuspid aortic root (i.e., only two nadirs) is encountered, the coplanar angiographic views can be calculated by incorporating the directional vector of the aortic root in conjunction with the two nadirs. A bifurcation is created automatically to facilitate the implant angiographic view computation where the first segment L^{Bicupid} is formed by connecting the two nadir points. The second segment L^{Aortic} (or branch) of bifurcation is followed by creating the segment passing through the midpoint of segment L^{Bicupid} with the directional vector of the aortic root and the same length of segment L^{Bicupid} The evaluation of the co-planar characteristic £²(α, β) can be done by calculating the degrees of foreshortening of the two segments based on a specific C-arm gantry angle as follows.

$${ℒ}^{2} ( α , β ) = \frac{{\vec{l}}^{Bicupid} \cdot \vec{{V}^{H}} ( α , β ) + {\vec{l}}^{Aortic} \cdot \vec{{V}^{H}} ( α , β )}{{L}^{B i c u p i d} + {L}^{A o r t i c}} , or$$

$${ℒ}^{2} ( α , β ) = \frac{{\vec{l}}^{Bicupid} \cdot \vec{{V}^{P}} ( α , β ) + {\vec{l}}^{Aortic} \cdot \vec{{V}^{P}} ( α , β )}{{L}^{B i c u p i d} + {L}^{A o r t i c}}$$

where “⋅” denotes the inner product operator, and V̅^̅{̅H̅}̅(α,β) and V̅^̅{̅P̅}̅(α, β) denote the unit normal vectors of image plane associated with the C-arm at head-side and physician-side locations defined by (α, β) gantry angles as follows.

$$\vec{{V}^{H}} ( α , β ) = [ \begin{matrix}sin β \\ - sin αcosβ \\ cos αcosβ\end{matrix} ] , \vec{{V}^{P}} ( α , β ) = [ \begin{matrix}cos αcos β \\ - sin α \\ cos αcosβ\end{matrix} ] .$$

Simply speaking, the value of £²(α, β) based on any specific C-arm gantry angles can be regarded as an implant angiographic view.

By use of the co-planar measurement £^{k}(α, β) the normalized function [symbol] _{COP}(α,β) with respect to the aortic cusps at the given C-arm gantry angle (α, β) is employed as

$${ℱ}_{C O P} ( α , β ) = \frac{{ℒ}^{k} ( α , β )}{{ℱ}_{C O P} ( \hat{α} , \hat{β} )} \times 100 \% , k = 1 , 2$$

where [symbol] _{COP}(α, β) denotes the maximum value of co-planar estimate at the specific gantry angle (\hat{α},\hat{β}) defined in the LAO-RAO and CRAN-CAUD angulation space. To best represent the degrees of co-planarity with respect to the C-arm gantry, a cusp co-planar map [symbol] _{COP}(α, β) is created where the horizontal axis and vertical axis of the map defines the C-arm gantry angle from LAO 60° to RAO 60° (i.e., −60≤α≤60) and CRAN 45° to CAUD 45° ((i.e., −45≤β≤60) respectively. The contents of [symbol] _{COP}(α, β) are assigned different gradient colors (i.e., white, green, yellow, blue, and red) representing various ranges of degrees of cusp overlap (i.e., 0%≤white color≤5%, 6%≤green color≤10%, 11%≤green color≤15%, 16%≤blue color≤20%, 21%≤red color≤100%).

Regarding Step 1440, which includes evaluating the degrees of cusp overlap, although there are multiple options selected from the coplanar curve, some of them may contain the projections with excessive cusp overlap where the calcifications at leaflet tips as landmarks for alignment of delivery system or coronary ostium are not visible adequately for coronary flow inspection on the cine angiography. It is necessary to evaluate the spatial relationships of aortic cusp on the projection view such that the projected cusp overlap can be minimized. The concept of Computer Graphics is employed to quantify cusp overlap. A image buffer I^{(a,b)} is allocated to simulate the projection of cusps based on any specific C-arm gantry defined by (a, b) (i.e., “a” denotes the LAO-RAO angles, and “b” denotes the CRAN-CAUD angles) where the set of polygons characterizing the surfaces of each cups are first projected onto the image buffer I^{(a,b)} followed by filled with a specific color (i.e., red for LCC, green for RCC, and blue for NCC). As a result, any pixel of image buffer with overlapped cusps may yield yellow (i.e., overlap between LCC and RCC), cyan (i.e., overlap between RCC and NCC), purple (i.e., overlap between LCC and NCC), or white (i.e., overlap among LCC, RCC, and NCC). By counting the numbers of pixels on image buffer with the overlapping color schemes, the degrees of cusp overlap can be accurately evaluated.

For example, there are multiple angiographic views that are qualified to serve as a coplanar working view for THV implant during the TAVR procedure. The main difference among the multiple coplanar views lies in the projected locations of individual cusps with different degrees of overlap. If excessive overlap between any two adjacent cusps are shown on the projection, some landmarks such as coronary ostium, calcifications may not be easily tracked or visualized for their shape and location changes subject to the force during the deployment of a THV. The present technique is able to evaluate the degrees of projected cusp overlap with respect to the C-arm gantry angulation.

Let T^{LCC}, T^{RCC}, and T^{NCC} denote the respective sets of triangles representing the 3D surface patches derived from the 3D structured models of aortic cusps P^{LCC}, P^{RCC} and P^{NCC} that are segmented from CT images as described earlier. Let T^{H}(α, β) and T^{P}(α, β) denote the transformation matrices associated with head-side and physician-side of C-arm configuration, respectively, where α and β denote the LAO-RAO and CRAN-CAUD angles of C-arm gantry. Let Tⱼ^{k}=(P_{j,1}^{k}, P_{j,2}^{k}, P_{j,3}^{k}), denote the three vertices of 3D triangle j where j=1,n corresponding to the number of triangles in the associated aortic cusp, and k is one of {LCC, RCC, NCC}. Based on the transformation matrix, the 2D projection vertices of each triangle T̅ⱼ^{k}(α, β)=(P̅_{j,1}^{k}, P̅_{j,2}^{k}, P̅_{j,3}^{k}), in each triangle can be calculated as follows.

$${\overline{T}}_{j}^{k} ( α , β ) = ( {\overline{P}}_{j , 1}^{k} , {\overline{P}}_{j , 2}^{k} , {\overline{P}}_{j , 3}^{k} ) = ( [ \begin{matrix}{\overline{P}}_{j , 1}^{k} ( x ) / {\overline{P}}_{j , 1}^{k} ( z ) \\ {\overline{P}}_{j , 1}^{k} ( y ) / {\overline{P}}_{j , 1}^{k} ( z )\end{matrix} ] , [ \begin{matrix}{\overline{P}}_{j , 2}^{k} ( x ) / {\overline{P}}_{j , 2}^{k} ( z ) \\ {\overline{P}}_{j , 2}^{k} ( y ) / {\overline{P}}_{j , 2}^{k} ( z )\end{matrix} ] , [ \begin{matrix}{\overline{P}}_{j , 3}^{k} ( x ) / {\overline{P}}_{j , 3}^{k} ( z ) \\ {\overline{P}}_{j , 3}^{k} ( y ) / {\overline{P}}_{j , 3}^{k} ( z )\end{matrix} ] ) ,$$

$$[ \begin{matrix}{\hat{P}}_{j , r}^{k} ( x ) \\ {\hat{P}}_{j , r}^{k} ( y ) \\ {\hat{P}}_{j , r}^{k} ( z )\end{matrix} ] = {T}^{H} ( α , β ) \cdot [ \begin{matrix}{P}_{j , r}^{k} ( x ) \\ {P}_{j , r}^{k} ( y ) \\ {P}_{j , r}^{k} ( z )\end{matrix} ] or$$

$$[ \begin{matrix}{\hat{P}}_{j , r}^{k} ( x ) \\ {\hat{P}}_{j , r}^{k} ( y ) \\ {\hat{P}}_{j , r}^{k} ( z )\end{matrix} ] = {T}^{P} ( α , β ) \cdot [ \begin{matrix}{P}_{j , r}^{k} ( x ) \\ {P}_{j , r}^{k} ( y ) \\ {P}_{j , r}^{k} ( z )\end{matrix} ] , r = 1 , 2 , 3$$

$$where$$

$${T}^{H} ( α , β ) = [ \begin{matrix}cos β & sin α sin β & - cos α sin β \\ 0 & cos α & sin α \\ sin β & - sin α cos β & cos α cos β\end{matrix} ]$$

$${T}^{P} ( α , β ) = [ \begin{matrix}cos β & 0 & - sin β \\ sin α sin β & cos α & sin α cos β \\ cos α sin β & - sin α & cos α cos β\end{matrix} ]$$

and (\hat{P}_{j,r}^{k}(x), \hat{P}_{j,r}^{k}(y), \hat{P}_{j,r}^{k}(z)) denote the j-th 3D coordinates of the vertex after transformation from the 3D point (P_{j,r}^{k}(x), P_{j,r}^{k}(y), P_{j,r}^{k}(z)) defined in the 3D model based on the C-arm gantry in term of transformation matrix T^{H}(α, β) or T^{P}(α, β).

By use of the projected 2D coordinates of triangle T̅ⱼ^{k}(α, β)=(P̅_{j,1}^{k}, P̅_{j,2}^{k}, P̅_{j,3}^{k}) consisting of three vertices, the triangle area is then filled with a pre-defined value by use of a “scan line polygon filling” algorithm in Computer Graphics such that regions resulting from the overlaps between any two cusps or all of the three cusps can be uniquely identified. A 3-bit pattern is used to store the required color to fill the area for each aortic color in conjunction with a matrix as computer memory to simulate the image plane. Specifically, the LCC, RCC, and NCC are assigned the values (001), (010), and (100), respectively. If overlap resulting from projection of cusps occur, the value (011), (101), (110), or (111) occurs on the pixel of simulated image plane [symbol] , which is different from the projected non-overlapping cusp region. Of course, the width of the bit pattern can be extended as needed if more than three objects are projected on to the image plane for assessment of degrees of overlap.

The percent of cusp overlap [symbol] _{COV} with respect to the aortic cusps at the given C-arm gantry angle (α,β) can be defined as

$${ℱ}_{C O V} ( α , β ) = \frac{{Σ}_{i = {x}_{min}}^{{x}_{max}} {Σ}_{j = {y}_{min}}^{{y}_{max}} ℘ ( i , j ) min}{{ℱ}_{COP} ( \hat{α} , \hat{β} )} \times 100 \% ,$$

$$℘ ( i , j ) = \{ \begin{matrix}1 , ( 011 ) , ( 101 ) , ( 110 ) or ( 111 ) \\ 0 , all other values\end{matrix} \} ,$$

where [symbol] (i,j) denotes the pixel value at location (i,j) on the simulated image plane [symbol] , (xₘᵢₙ, xₘₐₓ), (yₘᵢₙ,yₘₐₓ) denote the size of the image plane at respective horizontal and vertical directions, and [symbol] _{COP}(\hat{α},\hat{β}) denotes the maximum value of the cusp overlapping estimate at the specific gantry angle (\hat{α}, \hat{β}) defined in the LAO-RAO, and CRAN-CAUD angulation space. To best represent the degree of cusp overlap with respect to the C-arm gantry, a cusp overlap map [symbol] _{COP}(α,β) is created where the horizontal axis and vertical axis of the map defines the C-arm gantry angle from LAO 60° to RAO 60° (i.e., −60≤α≤60) and CRAN 45° to CAUD 45° (i.e., −45≤β≤60) respectively. The contents of [symbol] _{COV}(α, β) are assigned different gradient colors (i.e., white, green, yellow, blue, and red) representing various ranges of degrees of cusp overlap (i.e., 0%≤white color≤10%, 11%≤green color≤30%, 31%≤green color≤50%, 51%≤blue color≤70%, 71%≤red color≤100%).

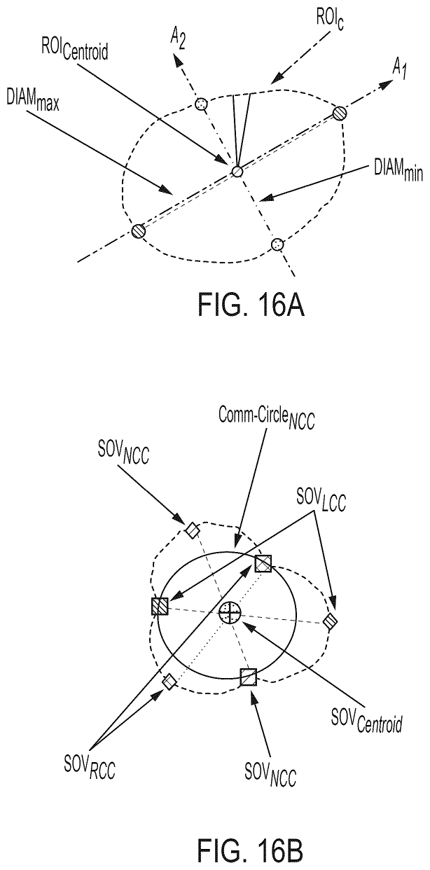

Additionally or alternatively, after the three nadir points are determined (for example, via the computational or manual methods described above), a method for determining an appropriate THV size may be employed. Such a method may include uniquely determining the virtual basial ring plane VBR_{Ω}. The intersection between the VBR_{Ω} and the 3D model near the LVOT region results in the annular contour Annu_{C} passing through the three nadirs. Similarly, the LVOT plane LVOT_{Ω} is determined by descending the BVR_{Ω} downward to the ventricular site by about 3 or 4 mm where the intersection between the LVOT_{Ω} and LVOT region results in the LVOT contour LVOT_{C}. The remaining contours for the the aortic geometry facilitating TAVR estimates are as follows: (a) ascending the BVR_{Ω} plane toward the ascending aorta (AO) region until passing through the commissure joint of three leaflets yielding the SOV plane SOV_{Ω}; (b) continue ascending the BVR_{Ω} plane until passing through the LCA and RCA ostial point resulting in the STJ plane STJ_{Ω} plane; (c) the ascending aorta plane is determined by ascending the STJ_{Ω} plane about 5 mm toward the aorta arch direction and results in the AO_{Ω} plane. Based on the derived Planex where x={Annulus, LVOT, SOV, STJ, AO}, the intersections between the individual plane and the 3D aortic model yield a corresponding Contour_{X} as shown in FIGS. 16A and 16B.

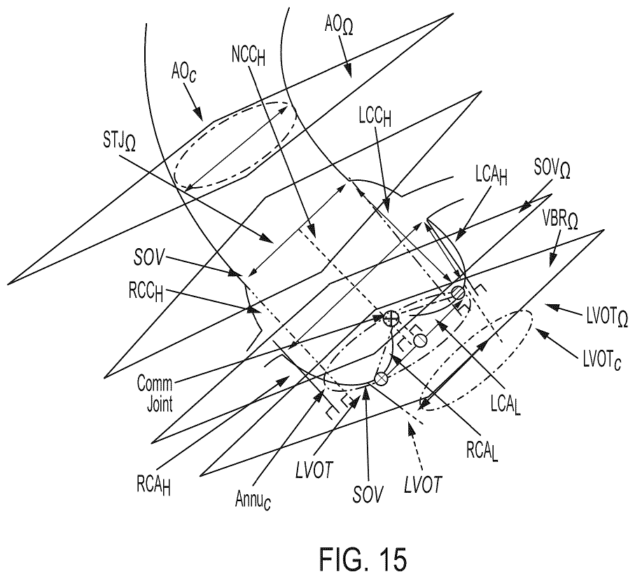

With the individual contours except the SOV contour generated, the minimum and maximum diameters, perimeter length, and area can be determined using the following steps: (A) By use of the Principal Component Analysis theory, the major A₁ and minor A₂ directional vectors are determined based on the set of points on the contour. (B) The two points on the contours that have maximum distances relative to the center of the contour along the A₁ and −A₁ directional vectors are determined where their distance is defined as the maximum diameter of the contour. (C) Similarly, the two points on the contours that have maximum distances relative to the center of the contour along the A₂ and −A₂ directional vectors are identified where their distance is regarded as the minimum diameter of the contour. (D) The perimeter of contour is calculated by adding every segment length formed by every pair of the two adjacent points on the contour. (E) The area of contour is calculated by adding every triangle area formed by two adjacent contour points and the contour centroid point. Due to the unique geometrical shape of SOV, it is evaluated in a different manner than the regular elliptical shape, as described above, using the following steps: (A) Identify the three (two) local points on the contours where the respective distance relate to the centroid point SOV_{Centroid} is a local maximum as shown as shown in FIG. 16B. (B) By use of the subset of points located within the two adjacent diamond symbols as identified in FIG. 16A, the point (i.e., joint between the adjacent cusps) can be identified if it has the shortest distance with respect to the centroid corresponding to the square symbol in FIG. 16B. (C) By connecting the pair of diamond and square symbols with the same color (or the longest distances), the three SOVx diameters can be defined where x={LCC, RCC, NCC}. (D) For a bicuspid aortic valve, the third pair of points or NCC landmarks will not be able to be identified and result in only two pairs of dominant (or LCC and RCC) landmarks where the method for a regular elliptical shape contour is used. Based on the aforementioned steps, the required estimates associated with contour-based region-of-interests (ROI) can all be derived. The remaining parameters mainly correspond to the linear distance-based estimate measured from the landmarks in the 3D model as follows (see FIG. 15): (A) The LCA/RCA ostial height (LCA_{H}/RCA_{H}) is determined by calculating the projection distance of the center point of the first cross section contour of LCA/RCA to the annular plane BVR_{Ω}. (B) The LCA/RCA leaflet length (LCA_{L}/RCA_{L}) is determined by calculating the distance between the associated LCC/RCC nadir to the commissure joint. (C) The LCC_{H}/RCC_{H}/NCC_{H} height is determined by calculating the projection distance of the respective LCC/RCC/NCC nadir point relative to the STJ_{Ω} plane.

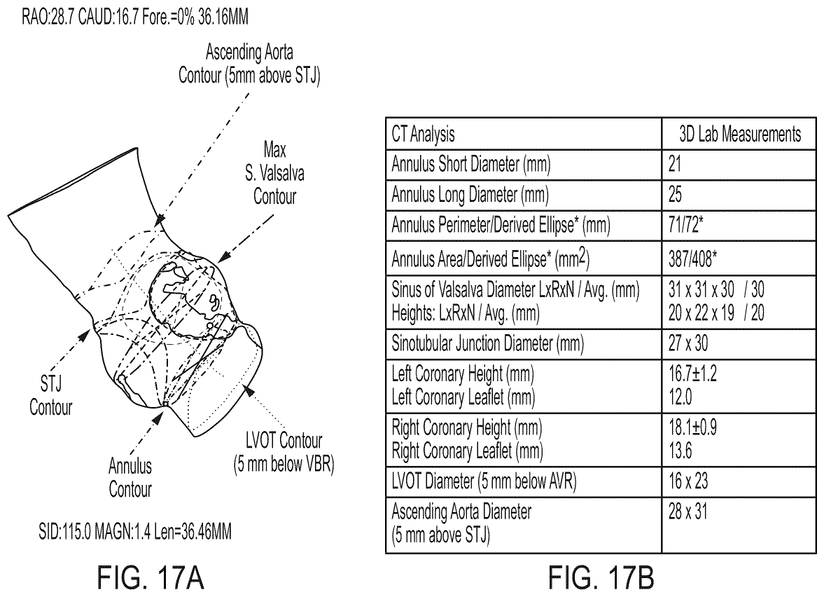

Referring to FIG. 17A, a typical 3D model of aortic root is illustrated where various contours corresponding to anatomical structures are identified first followed by calculations of the sizes and lengths among different region-of-interests. Referring to FIG. 17B, the estimated parameters may be tabulated as a list to facilitate selection of the type and the size of heart valve for a transcatheter-based interventional procedure.

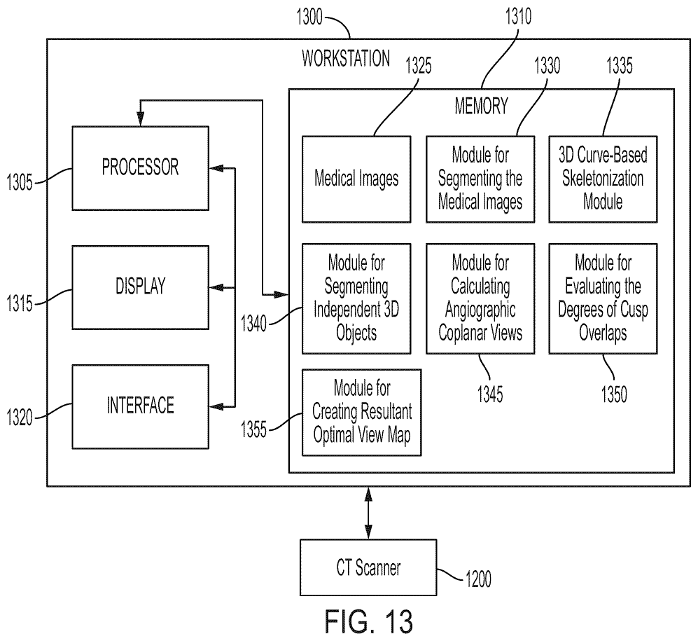

Referring to FIG. 13, there is depicted an illustration of a block diagram of an example workstation 1300 or computer system which may be used to implement all or certain or a combination of the methods illustrated in FIG. 14 and/or implement all or certain or a combination of aspects of the examples discussed herein. This diagram is merely an example, which should not unduly limit the scope of the claims. One of ordinary skill in the art would recognize many variations, alternatives, and modifications.

The workstation 1300 preferably includes a computer system comprising one or more processors 1305 and memory 1310 for storing programs and applications to perform the methods disclosed herein, as well as a storage unit, and/or a network interface. Memory 1310 may store a medical images module 1325, a module for segmenting the medical images 1330, a 3D curve-based skeletonization module 1335, a module for segmenting independent 3D objects 1340, a module for calculating angiographic views 1345, a module for evaluating the degrees of cusp overlap 1350, and a module for creating a resultant optimal view map 1355.

Medical images module 1325 may perform Step 1405, which includes receiving datasets of medical images. The module for segmenting the medical images 1330 may perform Step 1410, which includes segmenting the medical images to produce a set of 3D surface points. The 3D curve-based skeletonization module 1335 may perform Step 1415, which includes performing a 3D curve-based skeletonization process to transform the set of polygonal points generated from Step 1410 to a structure-based representation in terms of 3D skeleton or median curve. The module for segmenting independent 3D objects 1340 may be performed at Step 1420, which includes segmenting the individual coronary cusps and the extended aortic root (i.e., incorporation of the LVOT and proximal ascending aorta) as independent 3D objects with the structured point coordinates. The module for calculating angiographic views 1345 may perform Step 1425, which includes applying a parametric surface function to characterize each 3D cusp dataset, Step 1420, which includes a minimization process during which the 3D coordinates of a nadir point associated with each cusp can be calculated simultaneously, and Step 1435 includes using the nadirs to calculate the angiographic coplanar views resulting in a coplanar curve map. The module for evaluating the degrees of cusp overlap 1350 may perform Step 1440, which includes evaluating the degrees of cusp overlap. The module for creating a resultant optimal view map 1355 may perform Step 1445, which includes using of the information derived from the respective coplanar map and cusp overlap map.

The workstation 1300 may also include a display 1315 for viewing the images, map, and/or other resultants of the modules. Display 1315 may also permit a user to interact with the workstation 1300 and its components and functions (e.g., touchscreen, graphical user interface, etc.), or any other element within the system. This is further facilitated by an interface 1320 which may include a keyboard, mouse, a joystick, a haptic device, or any other peripheral or control to permit user feedback from and interaction with the workstation 1300. The workstation 1300 may also include or be coupled to the CT scanner 1200 or a PACS (not shown).

In some examples, the processor 1305 includes one or more general purpose microprocessors. In some examples, the main memory 1310 (e.g., random access memory (RAM), cache and/or other dynamic storage devices) is configured to store information and instructions to be executed by the processor 1305. In certain examples, the main memory 1310 is configured to store temporary variables or other intermediate information during execution of instructions to be executed by processor 1305. For example, the instructions, when stored in the storage unit accessible to processor 1305, render the computing system 1300 into a special-purpose machine that is customized to perform the operations specified in the instructions (e.g., the instructions stored in the components 300). In some examples, the ROM is configured to store static information and instructions for the processor 1305. In certain examples, the storage unit (e.g., a magnetic disk, optical disk, or flash drive) is configured to store information and instructions.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all the described features. Accordingly, the scope of the present disclosure is intended to embrace all such alternatives, modifications, and variations as fall within the scope of the claims, together with all equivalents thereof.

What is claimed is:

1. A non-transitory computer readable medium having a computer program stored thereon for performing a direct three-dimensional (3D) modeling technique for or in preparation for a transcatheter aortic valve replacement (TAVR) procedure, the computer program comprising instructions for causing one or more processors to:
receive a plurality of datasets of medical images comprising coronary anatomical structures;
segmenting the medical images to produce a set of 3D surface points, wherein the set of 3D surface points represents a 3D geometrical shape of the coronary anatomical structures;
performing a 3D curve-based skeletonization process to transform the set of 3D surface points to a structure-based representation of the coronary anatomical structures;
segmenting the structure-based representation of the coronary anatomical structures into independent 3D objects, wherein the independent 3D objects comprise at least two coronary cusps and an extended aortic root, wherein the at least two coronary cusps and the extended aortic root are each represented by a dataset of structured 3D point coordinates;
characterizing each dataset of structured 3D point coordinates as a parametric surface function;
performing a minimization process on the structured 3D point coordinates and the parametric surface functions to simultaneously determine 3D coordinates of nadir points associated with each of the least two coronary cusps;
using the 3D coordinates of the nadir points to calculate angiographic coplanar views and an associated cusp overlap map; and
deriving an optimal view map from the angiographic coplanar views and an associated cusp overlap map, the optimal view map comprising optimal viewing configurations for imaging equipment used during the TAVR procedure.

2. The non-transitory computer readable medium of claim 1, wherein the computer program further comprises instructions for causing one or more processors to evaluate a degree of overlap between the at least two cusps.

3. The non-transitory computer readable medium of claim 2, wherein the computer program further comprises instructions for causing one or more processors to use the degree of overlap between the at least two cusps to calculate the angiographic coplanar views and the associated cusp overlap map.
